(19) United States
(12) Patent Application Publication
(10) Pub. No.: US 2026/0334832 A1
(43) Pub. Date: Oct. 29, 2026

(54) SYSTEM FOR EVALUATING AND ASSESSING AN EMOTIONAL STATE OF A PET

(71) Applicant: ZPower Technology CO., LTD., Hsinchu (TW)

(72) Inventors: Shu-Jen CHEN, Hsinchu (TW); Yi-Lu TSAI, Hsinchu (TW); Yu-Syuan HUANG, Hsinchu (TW)

(21) Appl. No.: 19/647,241

(22) Filed: Apr. 14, 2026

(30) Foreign Application Priority Data
Apr. 25, 2025 (TW) 114115758

(51) Int. Cl. A61B 5/16; A01K 29/00; A61B 5/00; A61B 5/0205; A61B 5/024; A61B 5/087; A61B 5/11; A61B 8/02

(52) U.S. Cl. CPC A61B 5/165; A01K 29/007; A61B 5/0205; A61B 5/7203; A61B 5/7275; A61B 5/024; A61B 5/087; A61B 5/11; A61B 8/02

(57) ABSTRACT

A system for evaluating and assessing an emotional state of a pet, comprising a physiological sensing unit and a processing unit. The physiological sensing unit includes a non-optical heart rate sensor and a respiration sensor attached to the pet. The heart rate sensor outputs a first signal associated with a pulse and/or a heartbeat of the pet, and the respiration sensor outputs a second signal associated with respiration of the pet. The processing unit is connected to the physiological sensing unit, and receives the first signal to obtain heart rate and heart rate variability of the pet, and receives the second signal to obtain a respiration rate of the pet.

### CROSS-REFERENCE TO RELATED APPLICATIONS

This application claims priority of Taiwan Patent Application Number 114115758, filed on Apr. 25, 2025, the entirety of which is incorporated herein by reference.

### FIELD OF THE INVENTION

The present invention relates generally to a system for evaluating emotional states, and more particularly to a system for evaluating and assessing an emotional state of a pet.

### BACKGROUND OF THE INVENTION

With the development of society, an increasing number of people keep pets, which not only provide companionship in daily life but also help stabilize the owner's emotional well-being. Pet owners may attend to both physiological and psychological needs of pets, such as feeding and companionship. However, under current technological conditions, pets and humans are still unable to communicate through a common language. As a result, owners cannot accurately ascertain a pet's emotional condition, nor can they timely detect the pet's health status. Instead, owners must rely on routine behaviors or habitual patterns of the pet to infer its physiological and psychological conditions. Although professional assistance from veterinarians or trainers is available, such assistance is often constrained by time and location, making real-time support difficult to obtain.

Accordingly, there remains a need in the art for techniques capable of effectively enhancing interaction between pets and their owners, and for overcoming the limitations of existing approaches that are unable to provide real-time observation of a pet's emotional and health conditions.

### SUMMARY OF THE INVENTION

According to various embodiments of the present disclosure, a system for evaluating an emotional state of a pet is provided. The system comprises a physiological sensing unit and a processing unit. The physiological sensing unit comprises a non-optical heart rate sensor and a respiration sensor attached to a target pet. The heart rate sensor outputs a first signal associated with a pulse or heartbeat of the target pet, and the respiration sensor outputs a second signal associated with respiration of the target pet, the first signal and the second signal respectively representing heart rate data and respiration data of the target pet. The processing unit is coupled to the physiological sensing unit. The processing unit receives the first signal and the second signal to obtain the heart rate data and the respiration data. The heart rate data includes a heart rate and a heart rate variability of the target pet, and the respiration data includes a respiratory rate of the target pet.

The heart rate data and the respiration data are applied to an emotional condition classification model to classify the target pet into one of a high arousal/high valence state, a high arousal/low valence state, a low arousal/low valence state, or a low arousal/high valence state. The emotional condition classification model is represented as:

$${\hat{y}}_{i} = {\sum }_{k = 1}^{n} {f}_{k} ( {x}_{i} )$$

\hat{γ}ᵢ represents a predicted emotional score of an i-th sample, xᵢ represents an input feature vector, fₖ represents a k-th decision tree, and n represents a total number of decision trees, and the input feature vector is based on the heart rate data and the respiration data.

According to various embodiments of the present disclosure, a system for evaluating an emotional state of a pet is provided. The system comprises a physiological sensing unit, a motion sensing unit and a processing unit. The physiological sensing unit comprises a non-optical heart rate sensor and a respiration sensor attached to a pet. The motion sensing unit is attached to the pet. The processing unit is coupled to the physiological sensing unit and the motion sensing unit.

The system is configured to: obtain, by using the physiological sensing unit and the motion sensing unit, a physiological dataset of a plurality of labeled pets and an activity state dataset corresponding to the physiological dataset, wherein the physiological dataset comprises a plurality of physiological data, the physiological data comprising heart rate and respiratory rate associated with timestamps, and the activity state dataset comprises a plurality of activity state data, the activity state data temporally corresponding to the physiological data; determine whether the physiological dataset of each of the labeled pets includes one or more of noise; discard the noise from the physiological dataset; performing emotional labeling on the physiological data based on one or more images associated with the physiological data and containing the labeled pets; performing feature extraction on the physiological dataset to obtain a plurality of extracted features; performing feature fusion on the physiological dataset, wherein the feature fusion performs one or more operations on the extracted features to obtain a plurality of candidate features; performing feature selection on the candidate features, comprising: training a respective model based on each of the candidate features; fitting the model to obtain a residual between a predicted result and an actual result, wherein the actual result is based on the emotional labeling; and selecting one or more high-contribution features from the candidate features based on the residual; training a gradient boosting decision tree model using the high-contribution features as an input; performing cross-validation on the gradient boosting decision tree model; performing parameter optimization on the gradient boosting decision tree model; and obtaining an emotional classification model based on the gradient boosting decision tree model.

### BRIEF DESCRIPTION OF THE DRAWINGS

FIG. 1 is a schematic diagram of an embodiment of the present invention.

FIG. 2 is a schematic diagram of a system according to an embodiment of the present invention.

FIG. 3A is a schematic diagram of a modeling process according to an embodiment of the present invention.

FIG. 3B is a schematic diagram of an operation process according to an embodiment of the present invention.

FIG. 4 is a schematic diagram of an emotional quadrant according to an embodiment of the present invention.

FIG. 5 is a schematic diagram of an embodiment of the present invention.

### DETAILED DESCRIPTION

Terms used herein are for the purpose of describing particular embodiments only and are not intended to be limiting of the present invention. Unless the context clearly indicates otherwise, singular forms such as “a” and “the” include plural referents.

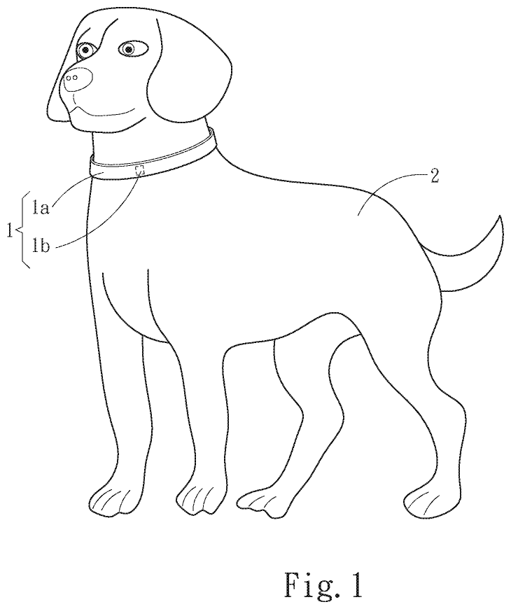

The present invention provides a system for detecting and evaluating an emotional condition of a pet. As illustrated in FIG. 1, the system includes a wearable device 1 configured to be worn on a body of a pet 2. The pet 2 may include, but is not limited to, a cat or a dog. The wearable device 1 includes a collar 1a and a sensor module 1b. The collar 1a is configured to position the sensor module 1b in proximity to or in contact with the body of the pet 2. The present invention is not limited to a neck-worn collar configuration, the collar 1a may be implemented as any suitable fastening structure or component capable of securing the sensor module 1b to the body of the pet 2. The pet 2 is likewise not limited to the example shown in FIG. 1.

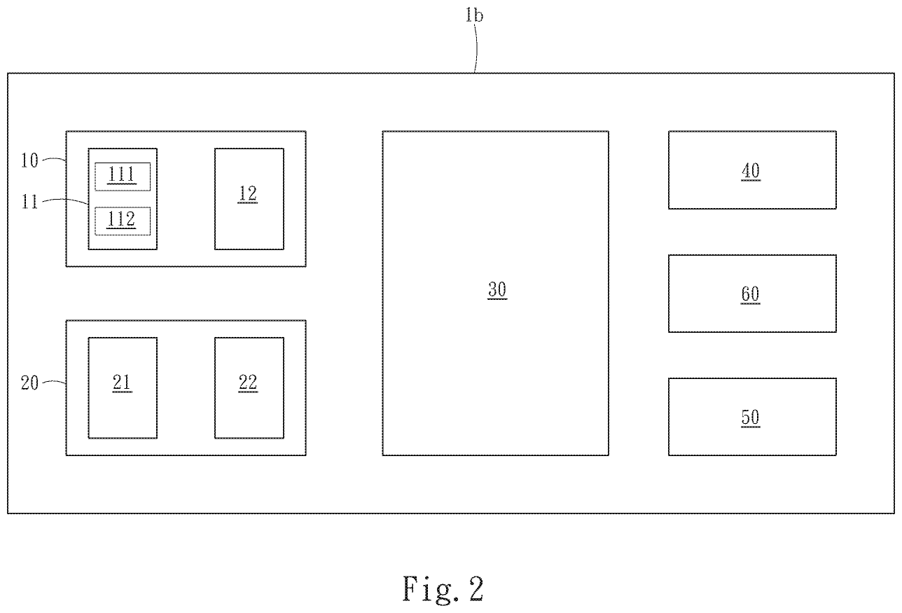

Referring to FIG. 2, the sensor module 1b includes a physiological sensing unit 10, a motion sensing unit 20, a processing unit 30, a storage unit 40, a power source unit 50, and a communication interface 60. In an example, the physiological sensing unit 10, the motion sensing unit 20, the processing unit 30, the storage unit 40, the power source unit 50, and the communication interface 60 may be disposed within a housing, and the housing is attached to the collar 1a.

The sensor module 1b is configured to acquire a heart rate and a respiratory rate of the pet 2, and to apply the data to an emotional classification model to generate a predicted emotional score. In one example, the physiological sensing unit 10, the motion sensing unit 20, the storage unit 40, and the communication interface 60 are coupled to the processing unit 30, and the processing unit 30 receives one or more sensing signals and data from the physiological sensing unit 10 and the motion sensing unit 20. In other examples, the physiological sensing unit 10 and the motion sensing unit 20 may be coupled to the storage unit 40, which receives and stores the sensing signals and data, and the processing unit 30 retrieves the sensing signals and data from the storage unit 40. The processing unit 30 may include a processor, a microprocessor, or the like. The storage unit 40 may include random access memory (RAM), dynamic random access memory (DRAM), static random access memory (SRAM), or other types of memory.

The power source unit 50 supplies electrical power to electronic circuits of the sensor module 1b, and may include a battery assembly. The communication interface 60 enables the sensor module 1b to transmit and/or receive data to and/or from other computers or devices. The communication interface 60 may include a communication chip configured to communicate using one or more communication protocols, such as WiFi, Bluetooth, 3G, 4G, 5G, or other communication standards. In some examples, the communication chip may include or be coupled to an antenna.

The physiological sensing unit 10 includes a heart rate sensor 11 and a respiration sensor 12, both configured to be attached to the body of the pet 2. In one example, the heart rate sensor 11 is a non-optical heart rate sensor and/or a non-contact heart rate sensor The heart rate sensor 11 outputs a first signal associated with a pulse and/or a heartbeat of the pet 2 to the processing unit 30, and the respiration sensor 12 outputs a second signal associated with respiration of the pet 2 to the processing unit 30. In the following description, “heart rate data” collectively refers to values associated with the pulse and/or heartbeat, such as beats per minute (BPM), and “respiration data” refers to respiration, such as breaths per minute. In one example, the first signal reflects or constitutes the heart rate data, and the second signal reflects or constitutes the respiration data.

In one example, the heart rate sensor 11 is an ultrasonic or millimeter-wave heart rate sensor, such as a patch-type ultrasonic heart rate sensor. The heart rate sensor 11 detects heart rate data of the pet 2 based on ultrasonic waves and/or millimeter waves, and may include a main body, one or more transmitters 111, and one or more receivers 112. The transmitters 111 and the receivers 112 may be disposed in the body and positioned on a side proximate to the body of the pet 2. By analyzing ultrasonic or millimeter waves reflected from the body of the pet 2, the pulse and heartbeat are detected.

The use of ultrasonic and/or millimeter-wave sensing avoids interference from fur on the skin of the pet 2, thereby enabling accurate acquisition of heart rate data. The heart rate data may be used to calculate or represent BPM, and based on successive heartbeat interval (RR interval) data, to compute a standard deviation of Normal-to-Normal intervals (SDNN), a root mean square of successive differences (RMSSD), and a ratio of low-frequency to high-frequency power (LF/HF), thereby reflecting autonomic nervous system activity of the pet 2. The respiration sensor 12 is configured to obtain the number of breaths per minute (respiratory rate) of the pet 2.

In one example, to track and compensate for individual differences and improve recognition accuracy, daily sampling may be performed for the pet 2. For example, the heart rate sensor 11 and the respiration sensor 12 may sample pulse, heartbeat, and/or breaths per minute of the pet 2 over a period of time within a day, such as a duration not less than one minute. The sampling results may be used to adjust or compensate measurements of the heart rate sensor 11 and the respiration sensor 12, and may also be used to adjust or compensate the emotional classification model.

The motion sensing unit 20 is configured to obtain an activity state of the pet 2, such as determining whether the pet 2 is stationary, walking, running, jumping, or engaging in other movements, and may further track trajectory, posture, or direction. The motion sensing unit 20 includes an accelerometer 21 and a gyroscope 22, configured to output a third signal and a fourth signal, respectively, to the processing unit 30. The third signal and the fourth signal are associated with the activity state. In one example, the accelerometer is a three-axis accelerometer, and the gyroscope is a three-axis gyroscope.

The processing unit 30 receives the first signal, the second signal, the third signal, and the fourth signal. Based on the first signal, the processing unit 30 obtains heart rate and heart rate variability (HRV) of the pet 2. Based on the second signal, the processing unit 30 obtains a respiratory rate. Based on the third signal and the fourth signal, the processing unit 30 obtains activity state data of the pet 2, such as movement direction, speed, frequency, range, intensity, and posture.

The storage unit 40 stores a database of pet-related emotional big data, including a real-time dataset and a historical dataset. The real-time dataset includes data associated with the first signal, the second signal, the third signal, and the fourth signal of the pet 2 at or near the present time, for example within a short time window (e.g., the past five minutes). The historical dataset includes emotional data measured and computed over a previous time period (e.g., a specific 15-minute interval from a prior day) associated with the pet 2 or other pet individuals.

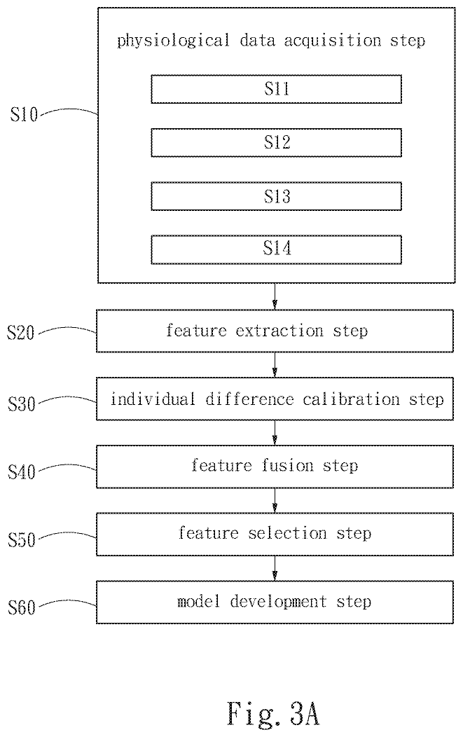

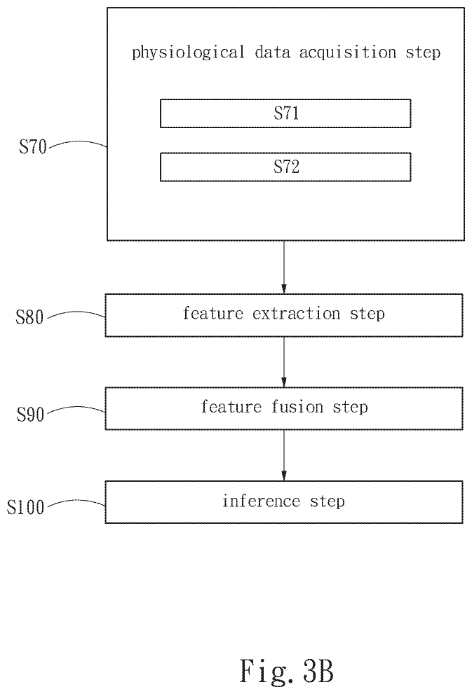

FIGS. 3A and 3B illustrate an example of flowchart. FIG. 3A relates to development of the emotional classification model, and FIG. 3B relates to use of the emotional classification model to the pet 2.

Referring to FIG. 3A, a physiological data acquisition step (step S10) is first performed. During a modeling phase, the wearable device 1 is deployed on multiple pets used for model training, which may be regarded as labeled or training pets. These pets may belong to the same or different species, for example all cats, all dogs, or a combination of cats and dogs. They may also belong to the same or different genera or families. The number of training pets may be on the order of tens, for example between 50 and 100, although more or fewer may be used depending on implementation.

In the physiological data acquisition step, data acquired by the sensor module 1b are first obtained, and the acquired data are filtered to retain data suitable for subsequent determination. The processing unit 30 may receive the first signal, the second signal, the third signal, and the fourth signal, thereby obtaining heart rate data, respiration data, and activity state data of the pet 2 (step S11). The heart rate and the respiratory rate are collectively referred to herein as physiological data. The physiological data and the activity state data may be acquired in real time or within a recent time window. For example, the physiological data and the activity state data may be continuously acquired over a first time interval to serve as model construction data. The first time interval may be, for example, within 30 minutes, such as 15 minutes, although longer or shorter intervals may be used in other examples. It should be understood that the physiological data are represented as a physiological dataset, and the activity state data are represented as an activity dataset. In one example, the physiological dataset includes heart rate and respiratory rate associated with timestamps, and the activity dataset includes activity state data associated with timestamps.

Subsequently, a data transformation step (step S12) is performed on the physiological dataset, in which numerical values of the physiological dataset are converted into a format suitable for subsequent algorithmic input.

Thereafter, a filtering step (step S13) is performed on the physiological dataset to remove motion-induced noise associated with the pet 2. For example, when the pet 2 is in a movement state, such as rapid running, the measured physiological data are affected by motion and are not solely attributable to the emotional condition of the pet 2. Accordingly, physiological data obtained while the pet 2 is in such a movement state are excluded in this step.

In one example, the filtering may include applying filtering operations to the third signal and the fourth signal, and selecting data within a defined range after filtering. For example, a high-pass filter may be applied to the third signal to obtain a plurality of first counting values, and a low-pass filter may be applied to the fourth signal to obtain a plurality of second counting values. In this example, the high-pass filtering may be performed at 1 Hz to capture high-frequency dynamic information, and the low-pass filtering may be performed at 3 Hz to remove high-frequency noise.

The third signal and the fourth signal may be processed using the following noise indicator to determine whether corresponding data are to be retained or discarded:

$$Noise indicator = \{ \begin{matrix}1 , & \sqrt[2]{{( {Gyr}_{x , y , z} )}^{2}} \geq T 1 , \sqrt[2]{{( {Acc}_{x , y , z} )}^{2}} \geq T 2 \\ 0 , & \sqrt[2]{{( {Gyr}_{x , y , z} )}^{2}} < T 1 , \sqrt[2]{{( {Acc}_{x , y , z} )}^{2}} < T 2\end{matrix}$$

Gyr_{x,y,z} represents the first counting values and may be regarded as gyroscope-related data associated with the third signal. Acc_{x,y,z} represents the second counting values and may be regarded as accelerometer-related data associated with the fourth signal. T1 and T2 are predefined threshold values, and may be distinct from each other. Data for which the noise indicator equals to one (1) are discarded, while only data for which the noise indicator equals to zero (0) are retained, representing stable signals.

In step S10, the physiological data are subjected to emotional labeling (step S14), which may be performed in conjunction with image data. For example, for a given pet, physiological data and one or more images of the pet may be acquired within the first time interval, each associated with corresponding timestamps. The physiological data and the images showing the pet are grouped into m sets according to the timestamps, with each group corresponding to a different timestamp (e.g., a first group corresponding to a first minute, a second group corresponding to a second minute, and so on). During acquisition of each group of physiological data, one or more imaging devices may be used to capture one or more images of the pet 2, which may include video or still images. Each group of physiological data is associated with one or more corresponding images, and the physiological data are labeled based on such images.

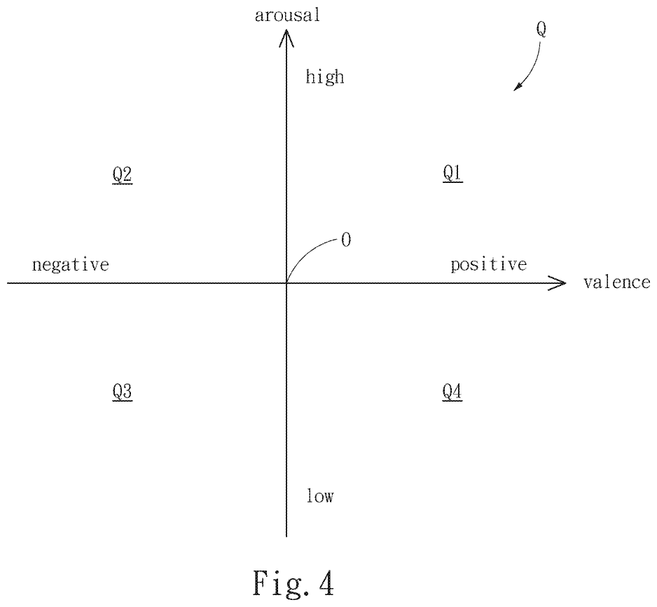

By way of example, for a particular pet, the physiological data and one or more images are acquired over the first time interval, each associated with a corresponding timestamp. Then, the emotional state of the pet 2 in the images is determined by a human annotator or by a software-based algorithm, and may be represented by an emotion quadrant. The emotional condition may be classified into the emotional quadrant, and may further be expressed by a corresponding emotional score. As illustrated in FIG. 4, the emotional state space Q includes a high arousal/high valence quadrant (HAHV) Q1, a high arousal/low valence quadrant (HALV) Q2, a low arousal/low valence quadrant (LALV) Q3, and a low arousal/high valence quadrant (LAHV) Q4, with a central region defined at and around point O, representing a neutral condition. The emotional score represents an intensity of emotion within a given quadrant. In other examples, the labeling may be performed in a non-manual manner, such as by image recognition techniques that label the emotional state of the pet 2 in the images corresponding to each group of physiological data.

An example of the labeling is shown in Table 1.

TABLE 1

| | Physiological | | Emotional | Emotional |
|---|---|---|---|---|
| Pet | Data Set | Image | Quadrant | Score |
| Pet 1 | 1 | P₁₁, P₁₂ | Q1 | A₁ |
| | 2 | P₂₁ | Q4 | A₂ |
| | 3 | P₃₁, P₃₂, P₃₃ | Q3 | A₃ |
| | . . . | . . . | . . . | . . . |
| | m | Pₘ₁ | Q2 | Aₘ |

The labeling may be performed on physiological data prior to filtering or on physiological data after filtering. It should be understood that the labeling results are utilized in subsequent steps, such as the two-stage pruning algorithm in step S50.

Step S20 is a feature extraction step. The physiological dataset reflects a variety of features, some of which have a substantial impact on emotional determination or model construction, while others have relatively lower contribution. The feature extraction step is to extract specific types of features from the physiological dataset that have been validated to exhibit high relevance.

The feature extraction includes a first feature extraction and a second feature extraction, which differ in terms of data source. The first feature extraction extracts features related to the heart rate data, excluding respiration-related features in this example. The second feature extraction extracts statistical features related to both the heart rate data and the respiration data.

Features related to heart rate data may include time-domain features, frequency-domain features, and/or nonlinear features. The time-domain features may be based on heart rate variability (HRV), including but not limited to HRV parameters, SDNN (standard deviation of NN intervals), and/or RMSSD (root mean square of successive differences between normal heartbeats). For example, HRV parameters may include pNN50, SDNN parameters may include NN50, and RMSSD parameters may include SD1. The frequency-domain features may be based on HRV, including but not limited to LF, HF,

$$\frac{LF}{HF} ,$$

arLF, arHF, and/or

$$\frac{arLF}{arHF} .$$

The nonlinear features may include, but are not limited to, SD2,

$$\frac{SD 1}{SD 2} ,$$

and/or SDRR.

The statistical features extracted in the second feature extraction include features related to both the heart rate data and the respiration data. Such statistical features may include, but are not limited to, mean, coefficient of variation, Rényi entropy, and/or Shannon entropy of the heart rate data and the respiration data. Features obtained from the first feature extraction and the second feature extraction may be collectively referred to as extracted features.

Step S30 is an individual difference calibration step. Normalization or standardization is performed based on a distribution of a historical dataset. The historical dataset may include physiological data and activity state data previously collected from multiple measurement subjects (e.g., pets of different species or different individuals of the same species), which are not the target subject currently being measured. In one example, the historical dataset may be the physiological dataset, or a combination of the physiological dataset and the activity dataset, of the aforementioned training pets. In this step, features of the historical dataset are subjected to a standard score transformation. The standard score transformation standardizes and maps physiological data and/or activity state data generated by different measurement subjects under emotional fluctuations (i.e., different predicted emotional scores) to a normal distribution having a mean of zero (0) and a standard deviation of one (1), such that the physiological data and/or activity state data of different measurement subjects are made comparable. In one example, the standard score transformation is calculated as: Z=(X−μ)/σ, where Z is the standard score, X is the original value, μ is the population mean, and σ is the population standard deviation. By calibrating based on the historical dataset of different measurement subjects, the prediction accuracy of the emotional classification model may be improved.

Step S40 is a feature fusion step, by which multiple candidate features are generated or obtained. The original physiological dataset is raw features, from which a set of derived features may be generated or obtained. The derived feature set may include, for example, expanded features, interaction features, transformed features, encoded features, deep features, and aggregated features. Among the derived features, some contribute more significantly to emotional determination, while others contribute less. The feature fusion step includes generating the derived feature set, evaluating contributions of the derived features, and selecting those with higher contributions as candidate features.

In this example, one or more feature engineering modules may be used, such as an OpenFE module. The extracted features obtained in step S20 are input into the OpenFE module, and one or more operations are performed using predefined operators, such as addition, subtraction, multiplication, division, averaging, minimum, and maximum operations. Based on these operations, multiple candidate features are generated from the extracted features.

Subsequently, feature selection is performed in conjunction with a two-stage pruning algorithm (step S50). In this example, a model trained using unexpanded physiological data (also referred to as original data) is referred to as an original model, and a model trained using expanded physiological data (also referred to as expanded data) is referred to as an expanded model. The two-stage pruning algorithm may adopt a coarse-to-fine approach. In a first stage, a first-stage algorithm is applied to train a model for each candidate feature individually, such that a respective model is trained for each candidate feature. Each model may be fitted to obtain a residual between a predicted result and an actual result, where the actual result is based on the emotional labeling performed in step S10. Based on the residuals of the respective models, a contribution (or weight) of each candidate feature to the model is evaluated, thereby identifying candidate features with low or negligible contribution in the first stage. In a second stage, the contribution values are used as input to a second-stage algorithm to further evaluate the utility weights of the candidate features.

In this example, the first-stage algorithm employs a FeatureBoost algorithm, and the second-stage algorithm employs a Forward Selection method. Through the two-stage pruning algorithm, candidate features of low importance are efficiently eliminated, and only those candidate features with high contribution to determining the emotional condition of the pet 2 are retained. These retained features are referred to as high-contribution features. In one example, such high-contribution features are as shown in Table 2.

TABLE 2

| High-Contribution Features | Data Source Category |
|---|---|
| Combined Rényi entropy and Shannon entropy | Heart rate data |
| Shannon entropy | Heart rate data |
| Minimum of RMSSD and pNN50 (min(RMSSD, pNN50)) | Heart rate data |
| Difference between heart rate and NN50 (HR-NN50) | Heart rate data |
| Root mean square of successive differences | Heart rate data |
| between adjacent NN intervals (RMSSD) | |
| Sum of heart rate and respiratory rate (HR + BR) | Heart rate data and |
| | respiration data |
| Respiratory rate (BR) | Respiration data |
| Standard deviation of the short axis of the Poincare | Heart rate data |
| plot (SD1) | |
| Standard deviation of the long axis of the Poincare | Heart rate data |
| plot (SD2) | |
| High-frequency component (HF) from spectral | Heart rate data |
| analysis reflecting parasympathetic activity | |

Finally, step S60 is a model development step, in which an emotional score prediction model is established using a regression algorithm. The emotional score prediction model is capable of handling individual variability, complex nonlinear relationships, and high-dimensional features. The model development step includes an initialization step, a cross-validation step, an optimization step, and a training step. The emotional score prediction model is developed using the high-contribution features obtained in step S50 as inputs.

In this example, a Gradient Boosting Decision Tree (GBDT) model is employed for training. In other words, the emotional score prediction model is based on a GBDT framework. The GBDT model may include, but is not limited to, LightGBM, XGBoost, and/or CatBoost. Upon introduction of the GBDT model, the model is first initialized, and the high-contribution features obtained in step S50 are used as inputs to the GBDT model.

Subsequently, a cross-validation scheme using a leave-one-subject-out approach is adopted to ensure that the parameters of the emotional score prediction model generalize across different subjects, such as cats, dogs, or other individual animals. In other words, the emotional score prediction model is applicable to predicting emotional conditions of previously unmeasured pet subjects.

Thereafter, an optimization step may be performed using a mean square error (MSE) criterion in conjunction with Bayesian optimization, to optimize a plurality of tree-specific parameters of the decision tree model. Such parameters may include, but are not limited to, the number of trees (n_estimators), maximum tree depth (max_depth), minimum number of samples required to split a node (min_samples_split), minimum number of samples required at a leaf node (min_samples_leaf), number of features considered when splitting a node (max_features), learning rate (learning_rate), and/or subsampling ratio (subsample).

The optimal parameter combination obtained in the optimization step is used to finalize the emotional score prediction model.

In this example, the emotional classification model is implemented as a gradient boosted decision tree regression model (e.g., an XGB regressor), and may be expressed as follows:

$${\hat{y}}_{i} = {\sum }_{k}^{n} {f}_{k} ( {x}_{i} )$$

\hat{γ}ᵢ represents a predicted emotional score of an i-th sample, xᵢ represents an input feature vector, fₖ represents a k-th decision tree, and n represents a total number of decision trees. The input feature vector xᵢ includes, but is not limited to, heart rate-related data and/or respiration-related data, such as heart rate, heart rate variability, and respiratory rate.

FIG. 3B illustrates a schematic flow of a pet emotional evaluation process in one example. A wearable device 1 is first mounted on a pet, which serves as a target subject for prediction. A physiological data acquisition step (step S70) is then performed to continuously obtain physiological data and activity state data of the target pet over a second time interval, thereby forming a dataset to be evaluated. The second time interval may be shorter than the first time interval, for example within 10 minutes, such as 5 minutes, although longer or shorter intervals may be used in other examples.

Next, a data transformation step (step S71) is performed on the physiological data to convert numerical values thereof into a format suitable for subsequent algorithmic input, with reference to step S12. Thereafter, a filtering step (step S72) is performed on the physiological data to remove motion-induced noise associated with the target pet, with reference to step S13.

Subsequently, a feature extraction step S80, a feature fusion step S90, and an inference step S100 are sequentially performed. The feature extraction step S80 and the feature fusion step S90 may refer to steps S20 and S40, respectively. In the inference step S100, the high-contribution features obtained from the feature fusion step S90 are input into the pre-established emotional classification model to obtain a prediction result, which is expressed as a predicted emotional score. The system may classify the target pet into a emotional quadrant based on the prediction result.

In certain examples, an individual difference calibration step, such as step S30, may be further included between the feature extraction step S80 and the feature fusion step S90. In such cases, the historical dataset used therein may correspond to a physiological dataset of the target pet, or a combination of the physiological dataset and the activity dataset. The collection period of the historical dataset may correspond to the second time interval, or may extend to an earlier or longer third time interval.

In certain examples, during pet emotional evaluation, activity state data of the pet 2 may be further derived from the third signal and used as an auxiliary indicator for emotional determination. For example, when the pet 2 is classified in the high arousal/high valence quadrant Q1 and exhibits a relatively active state, the pet 2 may be in an excited condition. Conversely, when the pet 2 is classified in the low arousal/low valence quadrant Q3 and exhibits a relatively inactive state, the pet 2 may be in a depressed or potentially unhealthy condition.

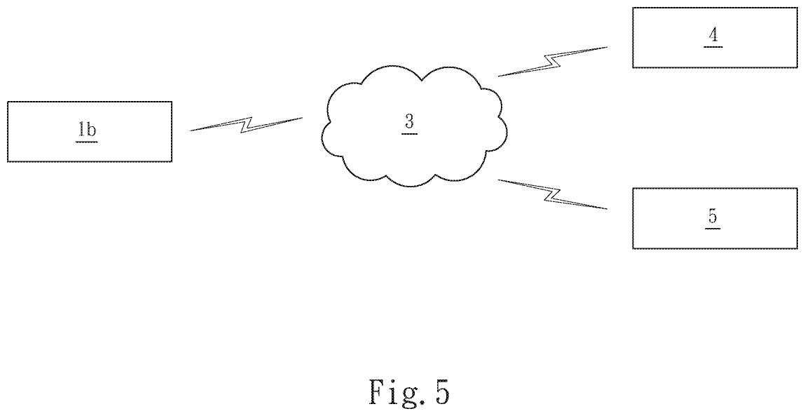

The present disclosure establishes a database and analysis methodology for heart rate variability of pets. The self-collected database enables effective characterization of distribution patterns of heart rate variability in pets, thereby improving the accuracy and reliability of emotional prediction. Referring to FIG. 5, the processing unit 30 may further transmit one or more types of data via the communication interface 60. The sensor module 1b may be connected to a network 3 via the communication interface 60, and further connected to a remote device 4, such as a server, a computing device, or other processing equipment, or to a user device 5 via the network 3, where the user device 5 may be a computer or a mobile device. In one example, the processing unit 30 uploads parameters such as heart rate, heart rate variability, respiratory rate, and activity state to the network 3 and the remote device 4 via the communication interface 60. The remote device 4 executes the emotional condition classification model to obtain the predicted emotional score, which is used to determine classification of the pet 2 into the emotional state space Q. The predicted emotional score, the classification result of the emotional state space Q, and the activity state are then transmitted to and displayed on the user device 5 for reference by a pet owner. In other embodiments, the processing unit 30 may locally execute the emotional condition classification model to generate the predicted emotional score, and transmit the predicted emotional score, the classification result, and the activity state to the user device 5 via the communication interface 60 for display.

In summary, the present disclosure utilizes the physiological sensing unit and the motion sensing unit to detect physiological parameters and activity conditions of a pet. Measured parameters including heart rate, heart rate variability, and respiratory rate are processed by the emotional condition classification model to generate a predicted emotional score, thereby enabling analysis of the emotional condition of the pet. The pet is classified into a corresponding emotional quadrant based on the predicted emotional score, and the activity state may be used in conjunction as a reference indicator for the pet owner to assess the pet's emotional condition.

What is claimed is:

1. A system for evaluating an emotional state of a pet, comprising:
a physiological sensing unit comprising a non-optical heart rate sensor and a respiration sensor attached to a target pet, wherein the heart rate sensor outputs a first signal associated with a pulse or heartbeat of the target pet, and the respiration sensor outputs a second signal associated with respiration of the target pet, the first signal and the second signal respectively representing heart rate data and respiration data of the target pet; and
a processing unit coupled to the physiological sensing unit, wherein the processing unit receives the first signal and the second signal to obtain the heart rate data and the respiration data, wherein the heart rate data includes a heart rate and a heart rate variability of the target pet, and the respiration data includes a respiratory rate of the target pet;
wherein the heart rate data and the respiration data are applied to an emotional condition classification model to classify the target pet into one of a high arousal/high valence state, a high arousal/low valence state, a low arousal/low valence state, or a low arousal/high valence state; and
wherein the emotional condition classification model is represented as:
$${\hat{y}}_{i} = {\sum }_{k = 1}^{n} {f}_{k} ( {x}_{i} )$$
wherein \hat{γ}ᵢ represents a predicted emotional score of an i-th sample, xᵢ represents an input feature vector, fₖ represents a k-th decision tree, and n represents a total number of decision trees, and the input feature vector is based on the heart rate data and the respiration data.

2. The system of claim 1, wherein the heart rate sensor detects the pulse or heartbeat of the target pet based on ultrasound and/or millimeter-wave signals, and comprises a main body, one or more transmitters, and one or more receivers disposed in the main body.

3. The system of claim 1, further comprising a motion sensing unit coupled to the processing unit, wherein the motion sensing unit outputs one or more third signals based on movement of the target pet, and the third signals are configured to obtain activity state data of the target pet.

4. The system of claim 3, wherein the system is configured to:
obtain, via the physiological sensing unit and the motion sensing unit, a physiological dataset and a corresponding activity dataset of a plurality of labeled pets, wherein the physiological dataset comprises a plurality of physiological data including timestamp-associated heart rate and respiratory rate, and the activity dataset comprises a plurality of activity data temporally corresponding to the physiological data;
determine whether the physiological dataset of each labeled pet includes noise, wherein corresponding physiological data are identified as noise when the activity data satisfy:
$$\sqrt[2]{( \text{?} Gy {r}_{x , y z} ) {\text{?}}^{2}} < T 1 , \sqrt[2]{\text{?} {( Acc {\text{?}}_{x , y , z} )}^{2}} < T 2$$
wherein Gyr_{x,y,z} represents gyroscope-related data and Acc_{x,y,z} represents accelerometer-related data in the activity dataset, and T1 and T2 are independent predefined threshold values;
discard the noise from the physiological dataset; and
perform emotional labeling on the physiological data based on one or more images containing the labeled pets and associated with the physiological data.

5. The system of claim 4, wherein the system is further configured to:
perform feature extraction on the physiological dataset to obtain a plurality of first extracted features and a plurality of second extracted features, wherein the first extracted features are selected from a group consisting of HRV, SDNN, RMSSD, LF, HF, LF/HF, arLF, arHF, arLF/arHF, SD2, SD1/SD2, and SDRR, and the second extracted features are selected from a group consisting of mean, coefficient of variation, Rényi entropy, and Shannon entropy of the heart rate and the respiratory rate.

6. The system of claim 5, wherein the system is further configured to:
perform feature fusion on the physiological dataset to generate a plurality of candidate features based on operations applied to the first extracted features and the second extracted features;
perform feature selection on the candidate features, including:
training a respective model for each candidate feature;
fitting the model to obtain a residual between a predicted result and an actual result, wherein the actual result is based on the emotional labeling; and
selecting one or more high-contribution features from the candidate features based on the residual.

7. The system of claim 6, wherein the high-contribution features are selected from a group consisting of a combination of Rényi entropy and Shannon entropy of heart rate, Shannon entropy of heart rate, a minimum of a root mean square of successive differences between adjacent normal-to-normal (NN) intervals and a proportion of NN interval differences greater than 50 ms (pNN50), a difference between heart rate and a number of NN interval differences greater than 50 ms (NN50), the root mean square of successive differences between adjacent NN intervals, a sum of heart rate and respiratory rate, respiratory rate, a standard deviation along a short axis of a Poincaré plot of R-R intervals, a standard deviation along a long axis of the Poincaré plot of R-R intervals, and a parasympathetic activity (high-frequency component, HF) derived from spectral analysis of heart rate.

8. The system of claim 6, wherein the system is further configured to:
train a gradient boosting decision tree (GBDT) model using the high-contribution features as input;
perform cross-validation on the GBDT model;
perform parameter optimization on the GBDT model; and
obtain the emotional classification model based on the GBDT model.

9. A system for evaluating an emotional state of a pet, comprising:
a physiological sensing unit comprising a non-optical heart rate sensor and a respiration sensor attached to a pet;
a motion sensing unit attached to the pet; and
a processing unit coupled to the physiological sensing unit and the motion sensing unit;
wherein the system is configured to:
obtain, by using the physiological sensing unit and the motion sensing unit, a physiological dataset of a plurality of labeled pets and an activity state dataset corresponding to the physiological dataset, wherein the physiological dataset comprises a plurality of physiological data, the physiological data comprising heart rate and respiratory rate associated with timestamps, and the activity state dataset comprises a plurality of activity state data, the activity state data temporally corresponding to the physiological data;
determine whether the physiological dataset of each of the labeled pets includes one or more of noise;
discard the noise from the physiological dataset;
perform emotional labeling on the physiological data based on one or more images associated with the physiological data and containing the labeled pets;
perform feature extraction on the physiological dataset to obtain a plurality of extracted features;
perform feature fusion on the physiological dataset, wherein the feature fusion performs one or more operations on the extracted features to obtain a plurality of candidate features;
performing feature selection on the candidate features, comprising:
training a respective model based on each of the candidate features;
fitting the model to obtain a residual between a predicted result and an actual result, wherein the actual result is based on the emotional labeling; and
selecting one or more high-contribution features from the candidate features based on the residual;
training a gradient boosting decision tree model using the high-contribution features as an input;
performing cross-validation on the gradient boosting decision tree model;
performing parameter optimization on the gradient boosting decision tree model; and
obtain an emotional classification model based on the gradient boosting decision tree model.

10. The system of claim 9, wherein the high-contribution features are selected from a group consisting of a combination of Rényi entropy and Shannon entropy of heart rate, Shannon entropy of heart rate, a minimum of a root mean square of successive differences between adjacent normal-to-normal (NN) intervals and a proportion of NN interval differences greater than 50 ms (pNN50), a difference between heart rate and a number of NN interval differences greater than 50 ms (NN50), the root mean square of successive differences between adjacent NN intervals, a sum of heart rate and respiratory rate, respiratory rate, a standard deviation along a short axis of a Poincaré plot of R-R intervals, a standard deviation along a long axis of the Poincaré plot of R-R intervals, and a parasympathetic activity (high-frequency component, HF) derived from spectral analysis of heart rate.
